(19) United States
(12) Patent Application Publication
(10) Pub. No.: US 2026/0020761 A1
(43) Pub. Date: Jan. 22, 2026

(54) INSTRUMENTED SCLERAL LENS AND ASSOCIATED DEVICE, OPTIONALLY FITTED IN THE LENS, FOR MEASURING THE PUPIL DIAMETER OF AN EYE

(71) Applicant: INSTITUT MINES TELECOM, PALAISEAU (FR)

(72) Inventors: Jean-Louis DE BOUGRENET DE LA TOCNAYE, GUILERS (FR); Vincent NOURRIT, BREST (FR); Kevin HEGGARTY, LANRIVOARE (FR)

(21) Appl. No.: 18/875,120

(22) PCT Filed: Jun. 9, 2023
(86) PCT No.: PCT/EP2023/065536

§ 371 Date: Dec. 13, 2024

(30) Foreign Application Priority Data
Jun. 14, 2022 (FR) 2205749

(51) Int. Cl. A61B 3/11; A61B 3/00

(52) U.S. Cl. CPC A61B 3/112; A61B 3/0008

(57) ABSTRACT

A scleral lens the membrane incorporates/encapsulates at least one light source, preferably a VCSEL, that delivers at least one beam to the iris of an eye. The proportion of the light beam reflected by the iris depends on the openness of the pupil. This reflected beam is collected by an element encapsulated in the membrane of the lens and analyzed by a suitable device. The analysis may either be carried out entirely within the scleral lens, which then contains a photodetector and an electronic chip that will calculate the openness of the pupil by comparing digital data obtained from signals converted by the photodetector with a previously determined look-up table, or externally to the lens by a suitable device.

### TECHNICAL FIELD

The present invention relates to an instrumented contact lens, and more particularly to a scleral lens.

The lens according to the invention may be a completely autonomous system embedded on at least one eye of an individual.

The invention in particular aims to automatically measure the pupil diameter of an individual by means of a scleral lens incorporating all or some of the constituent electronic components of a pupillometer.

The scleral lens according to the invention has many applications, among which mention may be made of medical ones such as addictology, anesthesia, ophthalmology, neurology, psychology, pharmacology, resuscitation, or toxicology. Generally, the invention, which makes it possible to measure the size of a pupil, is applicable in every field in which cognitive processes of the human brain are measured.

### PRIOR ART

It is known that the pupil of a human subject is an indicator of the physiological or psychological state thereof.

It has thus in particular been found that the size of an individual's pupil varies as a function of many parameters of the environment and of external stimulations, such as ambient light level, absorption of drugs or analgesics, as well as the individual's state of awakeness or emotional state or his sensitivity to pain.

Pupil measurements may be made using a pupillometer configured to measure variations in pupil size or shape.

A pupillometer is a device allowing the size of the openness of the pupil to be measured at various stages (at rest, miosis, mydriasis) without bringing an object into contact with the eyeball.

Known pupillometers most often incorporate lighting and a camera. They allow, for each eye, images processed in real time to be collected, in particular to measure pupillary diameter and the amplitude and speed of contraction or dilation: [1].

A standard dynamic pupillometer may take up to 30 measurements per second and a high-speed pupillometer up to 200.

The complexity of the image processing increases or decreases depending on the nature of the information sought and on the ambient lighting conditions.

In general, lighting is by an infrared source that illuminates the cornea, thus allowing measurement in scotopic regime, i.e. in the dark.

The measurements as such may be performed by various instruments.

A Colvard pupillometer uses a direct viewing system with a reticule graduated in millimeters.

A pupillometer is often integrated with other ophthalmic measuring devices such as aberrometers or topographers, for example those marketed under the name OPD SCAN III (Nidek) or Wavelight® Topolyzer™ VARIO (Alcon). In general, the patient presses his face against a fixed part of the device so that his eyes are isolated from ambient light that could affect the measurement.

Certain pupillometers, such as the pupillometer proposed in patent EP2609852B1, implement a method that involves lighting the cornea or iris directly and measuring the reflected intensity, which allows pupillary diameter to be deduced.

This technique has the major drawbacks of requiring closure of the eye and the presence of a wired communication device placed on the eye, which although suitable in the context of surgical interventions are unsuitable for measurement under conditions of ocular mobility and of movement of an individual.

There is therefore a need to further improve existing pupillometers, in particular in order to allow an individual wearing the equipment required to measure the diameter of his pupil not to be constrained in his ability to look and move about, and preferably in order to potentially allow use while the individual is walking around, e.g. to allow measurement independently of eye movements.

One aim of the invention is to at least partially meet this need.

### SUMMARY OF THE INVENTION

To do this, the subject of the invention, according to a first alternative, is a scleral lens for measuring the diameter of a pupil of an individual's eye, comprising:
a membrane configured to cover the pupil, the iris and at least partially the sclera of the eye;
a light source encapsulated in the membrane, the light source being configured to emit a light cone or beam intended to diverge directly or indirectly toward the iris;
an electronic circuit, encapsulated in the membrane and comprising at least as components:
   at least one photodetector arranged to capture the beam emitted by the light source reflected from the surface of the iris,
   a microcontroller, connected to the photodetector and configured to convert into digital data electrical signals generated by the photodetector and to calculate, based on the converted digital data, the diameter of the pupil using a predetermined look-up table, and to encode it for transmission by wireless communication,
   an antenna for transmitting information relating to the calculated pupil diameter by wireless communication.

Preferably, the photodetector is a photodiode.

Advantageously, the antenna is configured to transmit the information via near-field communication (NFC).

According to one advantageous embodiment, the antenna is further configured to electrically recharge the light source and/or active components of the electronic circuit.

According to a second alternative, the invention relates to a scleral lens for measuring the diameter of a pupil of an individual's eye, comprising:
a membrane configured to cover the pupil, the iris and at least partially the sclera of the eye;
a light source, encapsulated in the membrane and configured to emit a light cone or beam intended to diverge directly or indirectly toward the iris;
an optical element, encapsulated in the membrane and arranged to capture the beam emitted by the light source reflected by the surface of the iris and to redirect toward the exterior one or more light beams for calculating the diameter of the pupil.

Here, and in the context of the invention, the expression “scleral lens” has its usual meaning, namely a contact lens of large diameter that, when being worn on the eye, is supported by the sclera of the eye and passes like a bridge above the cornea without touching it.

The scleral lens according to the invention may be rigid or hybrid (semi-rigid).

The optical element is preferably a diffractive and/or refractive optical element.

According to one advantageous embodiment, the diffractive optical element consists of a microlens array.

Advantageously, the microlens array is made up of off-axis Fresnel lenses.

According to another advantageous embodiment, the scleral lens comprises another light source configured to emit a light beam intended to be directed toward the exterior of the membrane in a direction away from the eye.

Preferably, the light sources emit in the infrared.

According to one advantageous variant of embodiment, each light source is a laser, preferably a vertical-cavity surface-emitting laser (VCSEL), or an edge-emitting laser diode.

Advantageously, the VCSEL is equipped with an optical system for shaping its beam.

In one advantageous configuration, the scleral lens comprises:
an interface, encapsulated in the membrane, for collecting and supplying electrical energy to the light source and the active components of the electronic chip, from outside the lens;
at least one electronic circuit, encapsulated in the membrane and configured to activate the light source and the active components of the electronic chip via the interface.

According to another advantageous embodiment, the scleral lens comprises a battery encapsulated in the membrane and connected to the interface, the battery being configured to be recharged via the interface and to electrically power the light sources and/or optoelectronic functions associated with the light sources, the electronic circuit being configured to activate the sources via the battery.

The invention also relates to a pupillometer, comprising:
at least one scleral lens according to the first alternative of the invention;
a data acquisition system, arranged remotely from the lens and configured to receive the information relating to the calculated pupil diameter transmitted by the lens antenna by wireless communication.

The invention also relates to a pupillometer, comprising:
at least one scleral lens according to the second alternative of the invention;
a carrier, intended to be fixedly positioned with respect to the face of the individual;
securely fastened to the carrier, at least one photodetector for detecting the light beams emitted by the optical element of the lens so as to measure the diameter of the pupil based on the angle of deviation measured by the detector.

According to one advantageous embodiment, the pupillometer comprises a plurality of photodetectors taking the form of a strip of photodiodes or a photodetector array, the columns of which are configured to detect the light beams and the rows of which are configured to make the detection by the columns independent of a horizontal movement of the eye.

Advantageously, the carrier is a mounting, intended to be worn on the face of the individual, such as a spectacle frame or augmented-reality headset or augmented-reality device.

According to another advantageous embodiment, the pupillometer comprises at least one detector, securely fastened to the carrier, wherein the at least one detector is configured to detect the position of the light beam of the lens directed toward the exterior so as to extract therefrom the angle of deviation with respect to normal of the gaze.

Thus, the invention essentially consists of scleral lens the membrane of which incorporates/encapsulates at least one light source, preferably a VCSEL, that delivers at least one beam to the iris of an eye. The proportion of the light beam reflected by the iris depends on the openness of the pupil. This reflected beam is collected by an element encapsulated in the membrane of the lens and analyzed by a suitable device.

The analysis may either be carried out entirely within the scleral lens, which then contains a photodetector and an electronic chip that will calculate the openness of the pupil by comparing digital data obtained from signals converted by the photodetector with a previously determined look-up table, or externally to the lens by a suitable device. In the first alternative, calculated diameter is communicated to the world outside the lens via a wireless communication protocol, preferably NFC. In the second alternative, the light signals are transmitted to the outside world by means of an optical element that will create one or more spots of light on detectors, such as photodiodes arranged at a given distance from the scleral lens.

By virtue of the lens according to the invention, the dimension of the pupil may be measured without conscious participation by the individual wearing the scleral lens, irrespectively of whether he is moving about or not and without the need for him to close his eyelid, as in certain prior-art techniques.

Furthermore, it is quick and simple to measure pupil diameter with a lens according to the invention, because it does not require image processing as in certain prior-art techniques.

The scleral lens according to the invention advantageously integrates energy resources, e.g. one or more batteries or harvesting means, allowing the embedded functions to operate. In respect of the first alternative, the lens preferably incorporates wireless communication means as described in [2], and activating/deactivating means and means for transferring data upstream from the lens to an acquiring/control system remote from the lens.

The acquiring/control system and the optional detection of the optical beam spots generated by the lens according to the second alternative are advantageously integrated into a mounting, such as a pair of glasses, a mixed-reality headset, etc. as described in patent application FR1903979, which allows information to be exchanged with the scleral lens and energy to be supplied by a remote source.

It is possible to integrate other functions into a scleral lens according to the invention, such as an oculometry function as described in patent application FR2000575, a refractometry function as described in patent application FR2203684, and/or functions allowing complete measurement of parameters describing the attitude of the eye.

Other advantages and features of the invention will become more clearly apparent on reading the detailed description of examples of implementation of the invention, which is given by way of non-limiting illustration, and with reference to the following figures.

### BRIEF DESCRIPTION OF THE DRAWINGS

FIG. 1 is a schematic perspective view of a scleral lens according to the prior art, taking the general form of a spherical cap.

FIG. 2 is a schematic cross-sectional view illustrating a scleral lens according to a first alternative of the invention, placed on an eye the pupil diameter of which it is sought to measure.

FIG. 3 is a schematic front view of an iris and pupil of an eye, illustrating the possible variation in diameter of the iris.

FIG. 4 is a schematic cross-sectional view illustrating a scleral lens according to one variant of the first alternative of the invention.

FIG. 5 is a schematic cross-sectional view illustrating a scleral lens according to a second alternative of the invention, placed on an eye the pupil diameter of which it is sought to measure.

FIG. 6 is a schematic view illustrating one example of a diffractive optical element implemented in the alternative of FIG. 5 using an off-axis Fresnel lens in four distinct positions.

FIG. 7 is a schematic view illustrating two configurations of measurement of the diameter of a pupil, employing the diffractive optical element implemented in the alternative of FIG. 5.

FIG. 8 is a schematic cross-sectional view illustrating a scleral lens according to one variant of the second alternative of the invention.

FIG. 9 is a schematic view of a pupillometer with a scleral lens according to the alternative of FIG. 5, the carrier being glasses incorporating at least one strip of photodetectors.

FIG. 10 is a diagram showing operation of a device for recharging by electromagnetic induction making it possible to recharge the deformable battery encapsulated in a scleral lens according to the invention.

FIGS. 11A and 11B are views showing successive steps of manufacture of a scleral lens according to the invention.

### DETAILED DESCRIPTION

Throughout the present patent application, the terms “internal” and “external” are to be understood with reference to a scleral lens being worn on an eye. Thus, the internal face designates the face of the lens making contact with the surface of the eye, while the external face designates the face making contact with the outside world.

Likewise, the terms “above”, “below”, “top” and “bottom” are to be understood with reference to a scleral lens being worn on an eye the optical axis of which is substantially horizontal.

Here, and in the context of the invention, the expression “optical axis of the eye” means an axis identified in clinical practice as the direction connecting a point light source, and the center of the luminous reflexes of the four refractive surfaces of the eye (anterior and posterior faces of the cornea, anterior and posterior faces of the crystalline lens).

It will be noted that the various elements according to the invention have been shown only for the sake of clarity and that they have not necessarily been drawn to scale.

FIGS. 2 and 4 show two distinct alternatives of a scleral lens 1 intended to be worn on the eye of an individual.

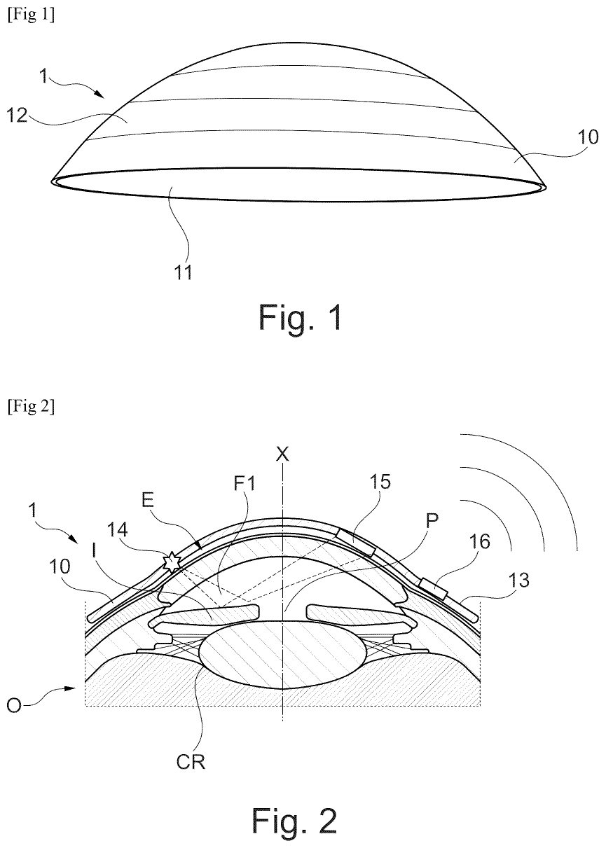

As illustrated in FIG. 1, a membrane 10 of a scleral lens 1 has an internal face 11, in particular the area bearing against the sclera, configured to achieve an optimum position and optimum stabilization on the eye, and an external face 12, these faces defining a general spherical-cap shape. Scleral lenses 1 do not make contact with the cornea, a space E (typically of a few hundred microns) being left between the surface of the cornea and the internal face 11 of the lens 1, the peripheral segment 13 of the lens 1 resting evenly on the sclera, as shown in FIGS. 2 and 4. These two features make this type of lens very comfortable, and very stable on the eye. The scleral lens 1 according to the first alternative, illustrated in FIG. 2, is configured to be applied to an eye O of an individual having an optical axis X.

The eye O has an iris I pierced at its center by a circular opening called the pupil P, through which light is transmitted. The iris I expands or contracts depending on luminous intensity. The eye O also comprises a crystalline lens CR formed by a transparent and flexible fibrous disc, for focusing incident light received through the pupil P.

As may be seen in FIG. 1, the pupil P and the crystalline lens CR are substantially centered on the optical axis X.

The scleral lens 1 bears, through encapsulation in its membrane 10, a light source 14. The central axis of the membrane 10 is substantially coincident with the optical axis X.

The transparent membrane 10 making contact with the cornea is preferably made of a biocompatible material, for example one based on silicone hydrogel or HEMA (acronym of hydroxyethyl methacrylate). Any other suitable biocompatible material may be used.

The source 14 may be a vertical-cavity surface-emitting laser (VCSEL) or an edge-emitting laser diode. The light emitted in the infrared by this source 14 may be coherent (VCSEL). Preferably, this source 14 is a VCSEL.

The fact that the scleral lens does not make direct contact with the cornea of the eye, because of the space E, thus allows the beam F1 of the source 14 to diverge sufficiently, and to illuminate a sufficient area of the iris I, for the diameter of the pupil P to be measured as detailed below. It may be envisioned to shape the source 14 (for example a diode of elliptical shape may be used) or to place beam-shaping optics on the source 14 so that the light beam F1 that the source emits illuminates the section of the iris I as best as possible, as described in patent application FR2203684.

A photodiode 15 is integrated into an electronic chip 16, also encapsulated in the membrane 10. As shown in FIG. 2, the photodiode 15 is arranged so as to capture the beam F1 emitted by the light source 14 after reflection from the surface of the iris I.

The electronic chip 16 comprises a microcontroller, which is connected to the photodetector by way of an analog-to-digital converter (ADC) and thus configured to convert the electrical signals generated by the photodetector into digital data. Thus, the microcontroller is able to calculate, based on the converted digital data, the diameter of the pupil using a predetermined look-up table.

The electronic chip 16 is further connected to an antenna (not shown) for transmitting data on the calculated pupil diameter via wireless communication, to the world outside the lens.

The wireless communication is preferably carried out according to an NFC protocol, NFC standing for near-field communication.

Thus, according to this first alternative, the measurement and the associated calculation of the diameter of the pupil P are entirely carried out within the scleral lens 1, only diameter-related information being transmitted externally, preferably via an NFC protocol, to a carrier such as a spectacle frame or to a cell phone, borne by the individual, or to a fixed external analyzer as described in [2].

Provision may be made for an inductive electrical recharging interface to be encapsulated in the membrane, with a view to collecting and supplying electrical energy to the light source 14 and the active components of the electronic chip 16, from outside the lens, at least one electronic circuit encapsulated in the membrane being configured to activate the light source and active components of the electronic chip via the interface.

Alternatively, the scleral lens may have integrated into its membrane 10 a rechargeable stand-alone battery that supplies the source and the active components of the chip 16 including the photodiode 15. This battery is advantageously an accumulator as described and claimed in patent application WO2018/167393A1.

The electronic chip 16 may for example be produced using a chip sold under the name NHS3152 by NXP.

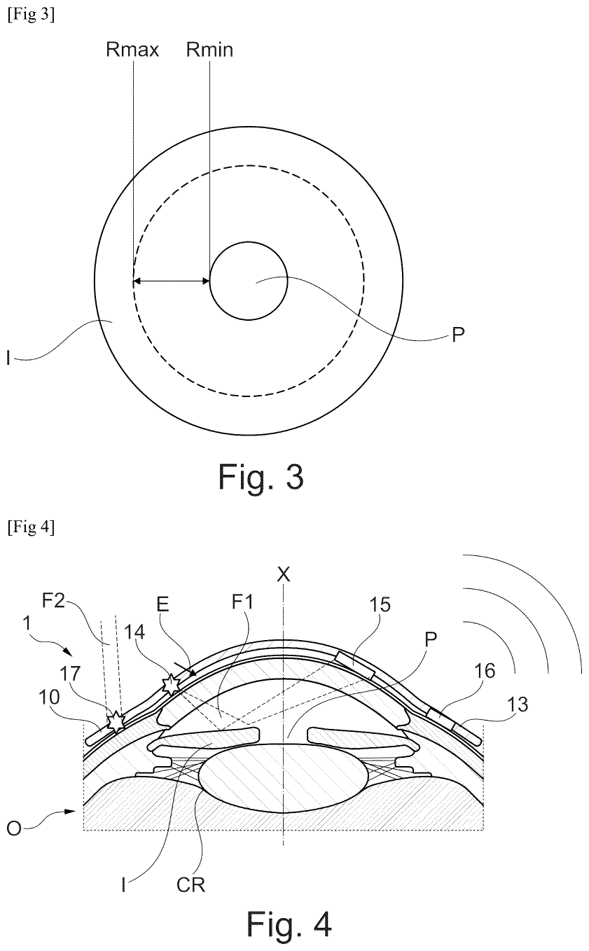

FIG. 3 schematically shows the iris I and pupil P of an eye O. Typically for a human being, the diameter of the pupil P may vary between about 2 and 8 mm. The light source according to the invention 14 must therefore illuminate a section of the iris I lying between Rmin and Rmax. For example, Rmin is equal to 1.5 mm and Rmax is equal to 3 mm. The look-up table may advantageously be determined based on these values.

In order to allow the light source 14 to illuminate the entire desired section of the iris I, it may be advantageous to lengthen the optical path traced by the beam F1 emitted by the source 14. For example, the light source 14 may be oriented toward the exterior and guided by multiple reflections as described in patent application FR2000575.

FIG. 4 illustrates a variant of the first alternative, in which a track of the direction of the gaze is closely monitored.

To do this, an additional light source 17 is encapsulated in the membrane 10.

The beam F2 emitted by this other source 17 illuminates toward the exterior, in order generate an optical track allowing the direction of the gaze to be determined. It is advantageously possible to form the beam F2 and detect it as described in patent application WO2020/212394

The light beam F2 may be analyzed by a position-sensitive detector (PSD) that may, depending on the configuration, be embedded in the lens and/or in a carrier that remains stationary with respect to the eye of the individual. Knowledge of the direction of the gaze makes it possible to compensate for the variable alignment of the eye with the PSD.

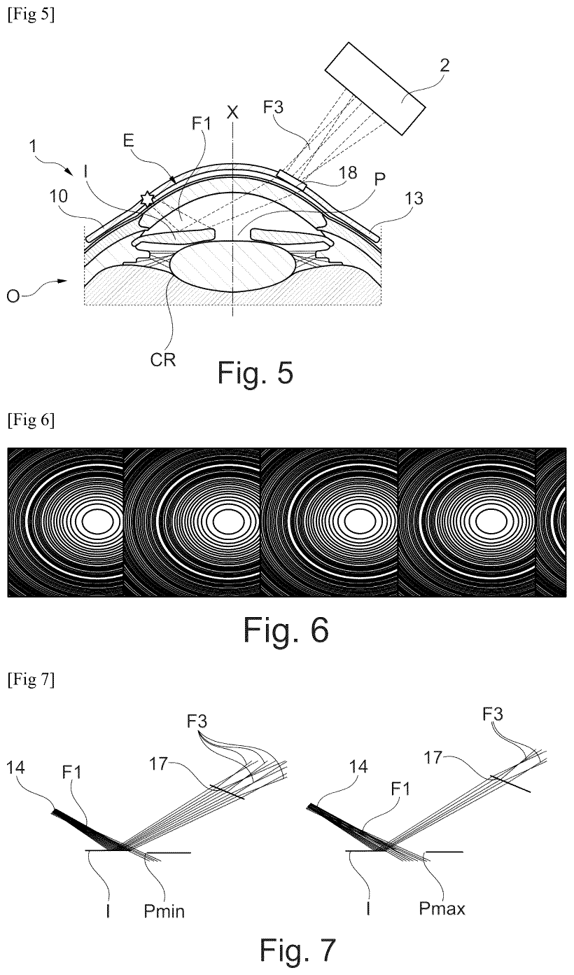

The scleral lens 1 in FIG. 5 implements a second alternative of the invention, in which the light beam F1 emitted by the light source 14 and reflected by the iris I is no longer analyzed within the lens itself (as in FIG. 2) but rather outside it by a detecting device 2 details of which are given below.

Thus, the lens 1 here comprises an optical element, preferably a diffractive optical element 18, that is encapsulated in the membrane 10 in an area facing the iris I, and preferably in an area facing the region where mydriasis occurs.

Depending on the percentage of the light beam F1 emitted by the source 14 that is reflected by the section of iris I, the optical element 18 will be illuminated to a greater or lesser extent.

The diffractive optical element 18 is made up of a number of facets, each giving rise to the creation, with a given optical power, of a light beam F3, which will form at a given distance from the lens 1, at which distance one or more detectors 2, and preferably one or more quadrant detectors, are arranged.

These detectors 2 may for example be quadrant photodiodes such as those sold by First Sensor: www.first-sensor.com/fr/produits/capteurs-optiques/detecteurs/apd-quadrants-qa/.

The detectors 2 preferably take the form of one or more strips of photodiodes.

Detection of each of the light beams F3 by the one or more detectors 2 located outside the lens 1 makes it possible to determine the diameter of openness of the iris.

The diffractive optical element 18 may advantageously consist of a microlens array, such as an array of prisms or a blazed grating (also called an echelette grating), or of a plurality of off-axis Fresnel lenses, i.e. Fresnel lenses each incorporating a deflection function so that each sector of the optical element 18 is imaged on a given photodiode of the strip of photodiodes, which given photodiode is different from those on which the other sectors of the optical element 18 are imaged. Since reflection from the semi-scattering surface of the iris has the effect of reducing the coherence of the light source 14, this type of component is highly suitable for creating the spots of light F3 in the desired manner.

FIG. 6 illustrates one example of a diffractive optical element 18 implemented in the alternative of FIG. 5 using an off-axis Fresnel lens, i.e. a Fresnel lens with a focus with an axial shift, in four distinct positions.

FIG. 7 illustrates two configurations of interception of the surface of the iris I by the beam F1 emitted by the light source 14, depending on the openness of the pupil (pupil open to the minimum Pmin on the left, and to the maximum Pmax on the right). The microlenses used as diffractive optical element 18 therefore here form 1 to 4 focal points depending on the size of the pupil and therefore on the area of iris I illuminated by the beam F1. It will be noted that, in FIG. 7, for the sake of simplicity, the cornea has not been shown: indeed, since the refractive indices of the aqueous humor, cornea and lacrimal fluid are close, being equal to 1.336, 1.376 and 1.33, respectively, the influence of the optical power of the cornea on the path traced is negligible, if the angle of incidence is not too far from normal.

The second alternative of the invention may also implement close monitoring of a track of the direction of the gaze.

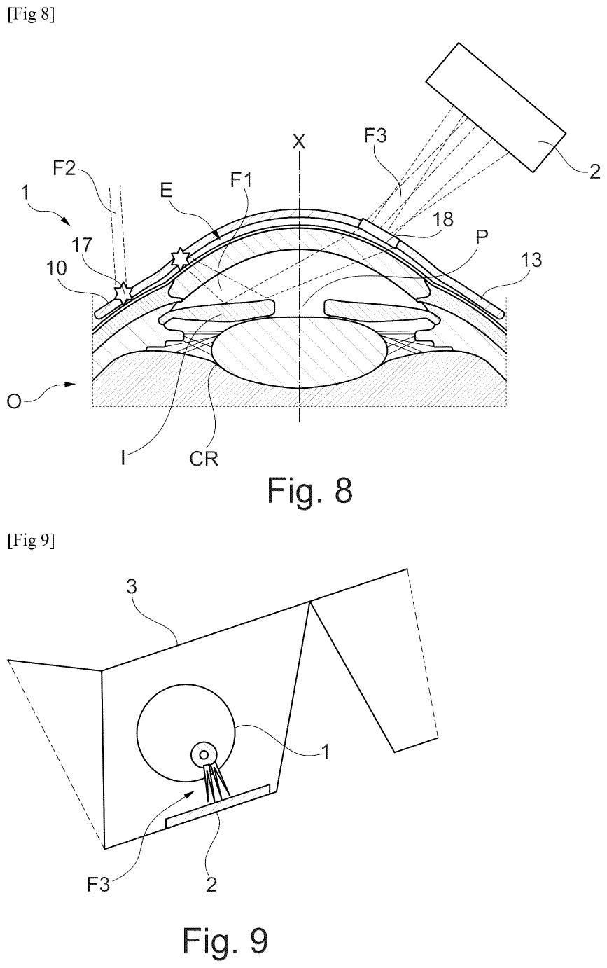

Thus, as illustrated in FIG. 8, an additional light source 17 is encapsulated in the membrane 10.

The beam F2 emitted by this other source 17 illuminates toward the exterior, in order generate an optical track allowing the direction of the gaze to be determined. It is advantageously possible to form the beam F2 and detect it as described in patent application WO2020/212394.

The light beam F2 may for example be analyzed by a position-sensitive detector (PSD) that may, depending on the configuration, be embedded in the lens and/or in a carrier that remains stationary with respect to the eye of the individual. Knowledge of the direction of the gaze makes it possible to compensate for the variable alignment of the eye with the PSD.

In general, no light source 14, 17 embedded in the lens 1 or any of the electronic components 15, 16 or optical element 18 embedded in the lens 1 blocks the sight of the individual.

The one or more detectors 2 for detecting the beams F3 are advantageously placed around the eye, preferably on a carrier worn by the individual, such as glasses 3, as schematically shown in FIG. 9. Typically, it may be a question of a strip of detectors 2, for example photodiodes arranged a few centimeters from the scleral lens 1 in the measurement configuration. The detection of the beams F3 on each of these detectors 2 thus makes it possible to determine the openness of the pupil P.

It is also possible to replace the strip 2 with an array of detectors 2 covering, for example, a given angular sector, typically about + or −15° around the scleral lens 1. Such an array also makes it possible to account for a horizontal movement of the eye O.

In the case where the scleral lens according to the invention has embedded in it a flexible battery for powering all the electronic/optoelectronic components, a system for recharging this battery by magnetic induction is advantageously provided. Thus, an antenna taking the form of an induction coil 19 connected to a rectifier is preferably encapsulated in the membrane 10 of a scleral lens 1.

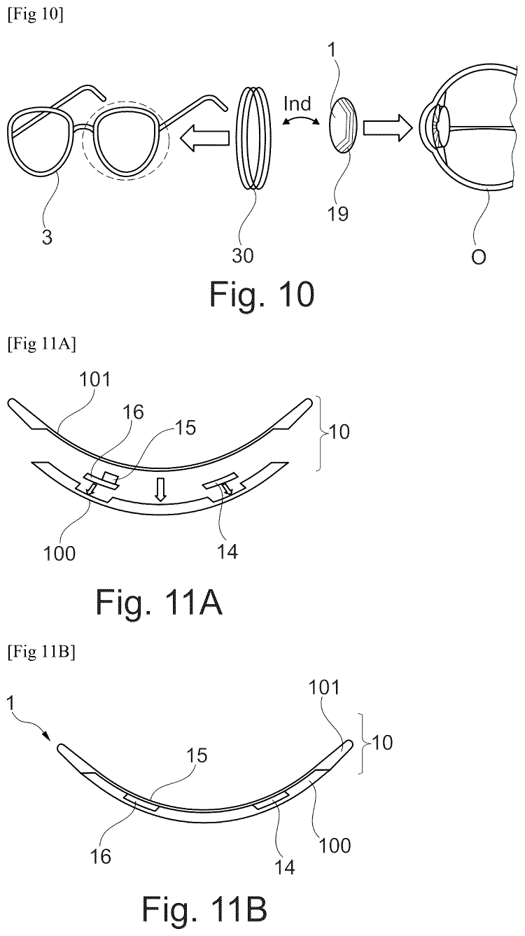

One advantageous example of a recharging system is shown in FIG. 10: an induction antenna 30 is integrated into a spectacle frame 3, which preferably bears the detectors 2 and, where appropriate, a position-sensitive detector (PSD) for detecting the beam F2. The antenna 30 transfers energy via magnetic coupling to the antenna 19 of the contact lens 1, which may be placed on the eye O of an individual during the recharging by magnetic induction. Reference may be made to publication [2] for further details.

FIGS. 11A and 11B illustrate certain steps of a process for manufacturing a scleral lens according to the first alternative of the invention.

The membrane 10 here consists of two films 100, 101 of transparent polymer, a hydrogel for example.

Each of the two films 100, 101 is first of all shaped as usual.

Next, all the electronics, with the possible exception of the antenna for collecting energy by induction, are placed on the interior face of the exterior film 100.

Thus the electronics, including the light source 14 and the electronic chip 16 with the photodiode 15, are perfectly positioned within the film 100.

Once this positioning has been carried out, the two transparent polymer films 100, 101 are sealed together, for example using UV adhesive.

Thus, all the electronic or optoelectronic components are perfectly positioned and encapsulated between the two films 100, 101.

The same method may be implemented to encapsulate the light source 14 and the diffractive element 18, in addition to the electronics.

Other variants and improvements may be made without however departing from the scope of the invention.

Although, in the examples illustrated, the photodetector 15 or the diffractive optical element 18 are arranged within the membrane 10 on the other side of the pupil P with respect to the light source 14, it is equally possible to envision arranging them side by side.

Likewise, although in the examples of FIGS. 4 and 8, the source 17 making it possible to determine the direction of the gaze is arranged side by side with the light source 14 that generates the beam that diverges toward the iris, it is also possible to envision placing it on the other side of the pupil P, for example beside the diffractive optical element 18.

Instead of or in addition to a diffractive element 18, it is possible to envision employing a refractive optical element.

### LIST OF CITED REFERENCES

[1] M. Larson, M. Behrends, “Portable Infrared Pupillometry: A Review”, Anesthesia and Analgesia 120 (6): 1242-53 DOI: 10.1213/ANE.0000000000000314, (2015).
[2] A. Khaldi, E. Daniel, L. Massin, C. Kärnfelt, F. Ferranti, C. Lahuec, F. Seguin, V. Nourrit, J-L de Bougrenet de la Tocnaye, “The cyclops contact lens: A laser emitting contact lens for eye tracking”, Scientific Report 10, 14804, doi.org/10.1038/s41598-020-71233-1, (2020).
[3] Applied Digital Optics: “From Micro-optics to nanophotonics (Chapter 6)”, Bernard C. Kress, Patrick Meyrueis, Wiley, November 2009, ISBN: 978-0-470-02264-1.

1. A scleral lens for measuring the diameter of a pupil of an individual's eye, comprising:
a membrane configured to cover the pupil, the iris and at least partially the sclera of the eye;
a light source encapsulated in the membrane, the light source being configured to emit a light cone or beam intended to diverge directly or indirectly toward the iris;
an electronic circuit, encapsulated in the membrane and comprising at least as components:
at least one photodetector arranged to capture the beam emitted by the light source reflected from the surface of the iris;
a microcontroller, connected to the photodetector and configured to convert into digital data electrical signals generated by the photodetector and to calculate, based on the converted digital data, the diameter of the pupil using a predetermined look-up table, and to encode it for transmission by wireless communication;
an antenna for transmitting information relating to the calculated pupil diameter by wireless communication.

2. The scleral lens as claimed in claim 1, wherein the photodetector is a photodiode.

3. The scleral lens as claimed in claim 1, wherein the antenna is configured to transmit the information via near-field communication.

4. The scleral lens as claimed in claim 1, wherein the antenna is further configured to electrically recharge the light source and/or active components of the electronic circuit.

5. A scleral lens for measuring the diameter of a pupil of an individual's eye, comprising:
a membrane configured to cover the pupil, the iris and at least partially the sclera of the eye;
a light source, encapsulated in the membrane and configured to emit a light cone or beam intended to diverge directly or indirectly toward the iris;
an optical element encapsulated in the membrane and arranged to capture the beam emitted by the light source reflected by the surface of the iris and to redirect toward the exterior one or more light beams for calculating the diameter of the pupil.

6. The scleral lens as claimed in claim 5, wherein the optical element is a diffractive and/or refractive optical element.

7. The scleral lens as claimed in claim 6, wherein the diffractive optical element consists of a microlens array.

8. The scleral lens as claimed in claim 7, wherein the microlens array is made up of off-axis Fresnel lenses.

9. The scleral lens as claimed in claim 1, comprising another light source configured to emit a light beam intended to be directed toward the exterior of the membrane in a direction away from the eye.

10. The scleral lens as claimed in claim 1, wherein the light sources emit in the infrared.

11. The scleral lens as claimed in claim 1, wherein each light source is a laser, preferably a vertical-cavity surface-emitting laser (VCSEL), or an edge-emitting laser diode.

12. The scleral lens as claimed in claim 11, wherein the VCSEL is equipped with an optical system for shaping its beam.

13. The scleral lens as claimed in claim 1, comprising:
an interface, encapsulated in the membrane, for collecting and supplying electrical energy to the light source and the active components of the electronic chip, from outside the lens;
at least one electronic circuit, encapsulated in the membrane and configured to activate the light source and the active components of the electronic chip via the interface.

14. The scleral lens as claimed in claim 13, comprising a battery encapsulated in the membrane and connected to the interface, wherein the battery is configured to be recharged via the interface and to electrically power the light sources and/or optoelectronic functions associated with the light sources, the electronic circuit being configured to activate the sources via the battery.

15. A pupillometer, comprising:
at least one scleral lens as claimed claim 1;
a data acquisition system, arranged remotely from the lens and configured to receive the information relating to the calculated pupil diameter transmitted by the lens antenna by wireless communication.

16. A pupillometer, comprising:
at least one scleral lens as claimed in claim 9;
a carrier, intended to be fixedly positioned with respect to the face of the individual;
securely fastened to the carrier, at least one photodetector for detecting the light beams emitted by the optical element of the lens so as to measure the diameter of the pupil based on the angle of deviation measured by the detector.

17. The pupillometer as claimed in claim 16, comprising a plurality of photodetectors taking the form of a strip of photodiodes or a photodetector array, the columns of which are configured to detect the light beams and the rows of which are configured to make the detection by the columns independent of a horizontal movement of the eye.

18. The pupillometer as claimed in claim 16, wherein the carrier is a mounting, intended to be worn on the face of the individual, such as a spectacle frame or augmented-reality headset or augmented-reality device.

19. The pupillometer as claimed in claim 15, comprising at least one detector, securely fastened to the carrier, wherein the at least one detector is configured to detect the position of the light beam of the lens directed toward the exterior so as to extract therefrom the angle of deviation with respect to normal of the gaze.
